(19) United States
(12) Patent Application Publication
(10) Pub. No.: US 2022/0399100 A1
(43) Pub. Date: Dec. 15, 2022

(54) MEDICAL IMAGE PROCESSING APPARATUS, MEDICAL IMAGE PROCESSING SYSTEM, AND NON-TRANSITORY COMPUTER READABLE STORAGE MEDIUM

(71) Applicant: CANON MEDICAL SYSTEMS CORPORATION, Otawara-shi (JP)

(72) Inventors: Haruki IWAI, Otawara (JP); Rie IKEZAKI, Sakura (JP); Naoko KURATOMI, Sakura (JP); Takahiro KUROKI, Nasushiobara (JP)

(73) Assignee: CANON MEDICAL SYSTEMS CORPORATION, Otawara-shi (JP)

(21) Appl. No.: 17/806,647

(22) Filed: Jun. 13, 2022

(30) Foreign Application Priority Data
Jun. 14, 2021 (JP) 2021-099032

(51) Int. Cl. G16H 30/20; G06F 3/0482; G06F 3/0484; G06T 7/00; G16H 50/20

(52) U.S. Cl. CPC G16H 30/20; G06F 3/0482; G06F 3/0484; G06T 7/0012; G16H 50/20; G06T 2207/30004; G06T 2200/24; G06T 2207/20092; G06T 2207/10116; A61B 6/5217

(57) ABSTRACT

The problem to be solved is to utilize an analysis result of a medical image that uses post-processing function to achieve a higher diagnosis accuracy. The medical image processing apparatus according to the present embodiment includes an acquirer that acquires a first medical image information which is a result of an external processing performed on a second medical information including a medical image, a receiver that receives a selected action for the first medical image information, and a register that associates and registers an action information about the selected action for the first medical image information with the first medical image information.

### CROSS REFERENCE TO RELATED APPLICATION

This application is based upon and claims the benefit of priority from the prior Japanese Patent Application No. 2021-099032, filed on Jun. 14, 2021, the entire contents of which are incorporated herein by reference.

### FIELD

Embodiments described in the present specification and drawings relate generally to a medical image processing apparatus, a medical image processing system, and a non-transitory computer readable storage medium.

### BACKGROUND

Recently, with the digitalization of X-ray images, post-processing function has improved, which outputs an analysis result, such as analyzing images from X-ray imaging apparatus and automatically detecting portions from the X-ray images with noticeable characteristics. A typical example which realizes this post-processing function is a Computer Aided Diagnosis (CAD) apparatus.

However, the analysis result of the CAD apparatus causes inconvenience to a user since the result cannot be confirmed in the X-ray imaging apparatus. Depending on the analysis result of the CAD apparatus there also may be situations that require an urgent treatment, which the user must be able to perform actions such as confirming the analysis result or notifying a medical personnel based on the analysis result of the CAD apparatus. However, it is hard to say that the action is well recorded or managed, since the current X-ray imaging apparatus fails to record a user action based on the analysis results of the CAD apparatus.

The same problem occurs not only in X-ray imaging apparatus such as a typical X-ray diagnosis apparatus and an X-ray Computed Tomography (CT) apparatus, but also in a medical imaging apparatus such as an ultrasonic diagnosis apparatus and a Magnetic Resonance Imaging (MRI) apparatus. If the information can be collectively managed, a convenience of the user and diagnosis accuracy will increase. Furthermore, for other medical image processing apparatus other than the medical imaging apparatus, the convenience of the user and diagnosis would also improve if the information can be collectively managed.

### BRIEF DESCRIPTION OF THE DRAWINGS

FIG. 1 is a block diagram illustrating an exemplary configuration of a medical image processing system according to a first embodiment.

FIG. 2 is a block diagram illustrating an exemplary configuration of a medical image diagnosis apparatus according to the first embodiment.

FIG. 3 is a flowchart for explaining a content of an action information registration and transmission process performed on a medical image processing apparatus according to the first embodiment.

FIG. 4 is a diagram illustrating an example of an analysis result data reception management list that indicates a destination of a medical image data and whether the analysis result data has been received according to the first embodiment.

FIG. 5A is a diagram illustrating an example of a list of the medical image data and the analysis result data displayed on a display according to the first embodiment.

FIG. 5B is a diagram illustrating an example of an action selection screen displayed on the display according to the first embodiment.

FIG. 6A is a diagram illustrating an example of an associated list of the action information and an analysis result data according to the first embodiment.

FIG. 6B is a diagram illustrating an example of a confirmation screen displayed on the display according to the first embodiment.

FIG. 7 is a diagram illustrating an example of a list of the analysis result data and action information associated with the list of analysis result data according to the first embodiment.

FIG. 8 is a flowchart for explaining a content of a notification process performed on the medical image processing apparatus according to the first embodiment.

FIG. 9 is a diagram illustrating an example of a condition list that defines a condition of the analysis result data to notify when an action has not been registered, in the medical image processing system according to the first embodiment.

FIG. 10 is a diagram illustrating an example of a notification screen displayed on the display of the medical image processing apparatus according to the first embodiment.

FIG. 11 is a flowchart for explaining an output process of a examination list, performed on the medical image processing apparatus according to the first embodiment.

FIG. 12 is a diagram illustrating an example of a examination list generated in the output process of the examination list of FIG. 11.

FIG. 13 is a flowchart for explaining a content of a search process performed on the medical image processing apparatus according to the first embodiment.

FIG. 14 is a diagram illustrating an example of a search list generated in the search process of FIG. 13.

FIG. 15 is a block diagram illustrating an exemplary configuration of the medical image processing system according to a second embodiment.

FIG. 16 is a block diagram illustrating an exemplary configuration of the medical image processing apparatus according to the second embodiment.

FIG. 17 is a block diagram illustrating an exemplary configuration of the medical image diagnosis apparatus according to the second embodiment.

FIG. 18 is a diagram illustrating an example of an image information included in the analysis result data.

FIG. 19 is a block diagram illustrating an exemplary configuration of the medical image processing system according to a modified example of the first embodiment.

FIG. 20 is a block diagram illustrating an exemplary configuration of the medical image processing system according to a modified example of the second embodiment.

### DETAILED DESCRIPTION

Hereinafter, respective embodiments of medical image processing systems will be described with reference to the accompanying drawings. In the embodiments below, the same reference signs are given for identical components in terms of configuration and function, and duplicate description is omitted.

### First Embodiment

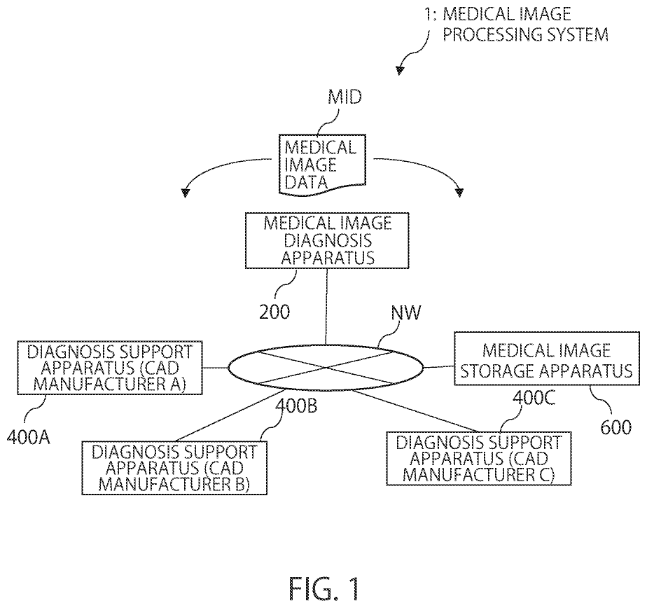

FIG. 1 is a block diagram illustrating an exemplary configuration of a medical image processing system 1 according to a first embodiment. As shown in FIG. 1, the medical image processing system 1 according to the first embodiment is configured with a medical image diagnosis apparatus 200, a diagnosis support apparatus (manufacturer A) 400A, a diagnosis support apparatus (manufacturer B) 400B, a diagnosis support apparatus (manufacturer C) 400C, and a medical image storage apparatus 600. The medical image diagnosis apparatus 200, the diagnosis support apparatus (manufacturer A) 400A, the diagnosis support apparatus (manufacturer B) 400B, the diagnosis support apparatus (manufacturer C) 400C, and the medical image storage apparatus 600 are connected via a network NW to communicate with each other. Hereinafter, when referring to without distinction, the diagnosis support apparatus (manufacturer A) 400A, the diagnosis support apparatus (manufacturer B) 400B, and the diagnosis support apparatus (manufacturer C) 400C will be simply referred to as a “diagnosis support apparatus 400”.

The medical image diagnosis apparatus 200 acquires a medical image data MID by imaging a subject. Then, the medical image diagnosis apparatus 200 transmits the acquired medical image data MID to the diagnosis support apparatus 400 or the medical image storage apparatus 600. The medical image diagnosis apparatus 200 may be an X-ray diagnosis apparatus, an X-ray imaging apparatus such as an X-ray CT apparatus, an ultrasonic diagnosis apparatus, or an MRI apparatus. The medical image data MID imaged by the medical image diagnosis apparatus 200 is equivalent to a second medical image information in the present embodiment.

The diagnosis support apparatus 400 performs an external processing on the medical image data MID and generates an external processing result. Specifically, the diagnosis support apparatus 400 may perform an image analysis on the medical image data MID and generate an analysis result data about a detected disease or infection, a discrimination of how good or bad the disease or infection is, or the like. Then, the diagnosis support apparatus 400 transmits the generated analysis result data to the medical image diagnosis apparatus 200 or medical image storage apparatus 600. The external processing result generated by the diagnosis support apparatus 400 is equivalent to a first medical image information in the present embodiment. Hereinafter, the present embodiment will be explained with reference to the analysis result data which is one example of the external processing result.

Note that there are three apparatus, the diagnosis support apparatus (manufacturer A) 400A, the diagnosis support apparatus (manufacturer B) 400B, and the diagnosis support apparatus (manufacturer C) 400C connected to the network NW in FIG. 1 of the present embodiment, but the number of the diagnosis support apparatus connected to the network NW is not limited to this example. For instance, one or two diagnosis support apparatus 400 may be connected to the network NW, or more than four diagnosis support apparatus 400 may be connected to the network NW.

The medical image storage apparatus 600 stores medical image data MID acquired by the medical image diagnosis apparatus 200, analysis result data generated by diagnosis support apparatus 400, information associated with the subject, or the like. Also, the medical image storage apparatus 600 transmits data such as stored medical information data MID, analysis result data, and information associated with the subject to the medical image diagnosis apparatus 200 or the diagnosis support apparatus 400. The medical image storage apparatus 600 may be an image server such as a Picture Archiving and Communication System (PACS).

The network NW is an information and communication network that uses telecommunications technology in general. The network NW may be a telecommunications network, a fiber optic communication network, a cable communication network, a satellite communication network, as well as an internet network or a wireless/wired local area network (LAN) such as a backbone network LAN of a hospital.

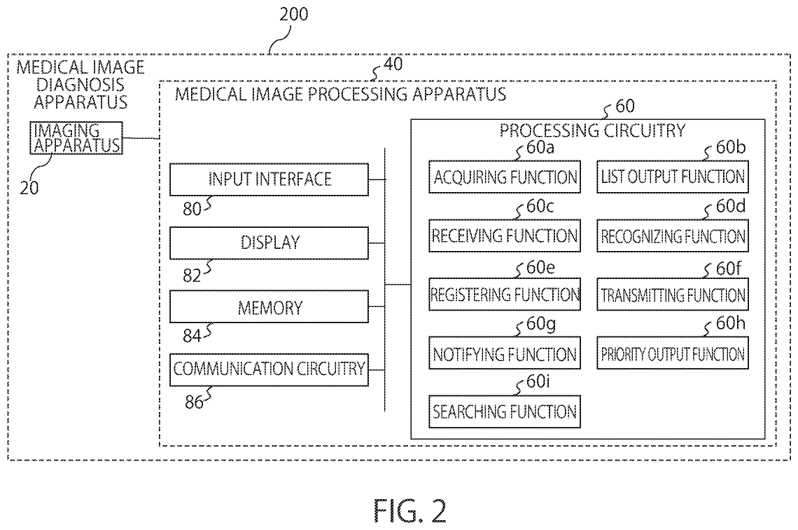

FIG. 2 is a block diagram illustrating an exemplary configuration of the medical image diagnosis apparatus 200 according to the first embodiment. As shown in FIG. 2, the medical image diagnosis apparatus 200 is configured with an imaging apparatus 20 and a medical image processing apparatus 40. The imaging apparatus 20 has an imaging mechanism to image the subject and is configured to acquire the medical image data MID of the subject. The medical image data MID of the subject imaged by the imaging apparatus 20 is processed in the medical image processing apparatus 40.

In the present embodiment, the medical image processing apparatus 40 is configured with a processing circuitry 60, an input interface 80, a display 82, a memory 84, and a communication circuitry 86. These multiple elements that configure the medical image processing apparatus 40 may be stored in a single housing or may be distributed into multiple housing as well.

The processing circuitry 60 is an arithmetic circuitry that performs various arithmetic operation. For instance, the processing circuitry 60 according to the present embodiment may not only process the medical image data MID imaged by the imaging apparatus 20, but also may acquire and display the analysis result data generated by the diagnosis support apparatus 400 or register a performed user action based on the analysis result data.

For this reason, the processing circuitry 60 has an acquiring function 60a, a list output function 60b, a receiving function 60c, a recognizing function 60d, a registering function 60e, a transmitting function 60f, a notifying function 60g, a priority output function 60h, and a searching function 60i. The acquiring function 60a is equivalent to an acquirer according to the present embodiment; the list output function 60b is equivalent to a list output according to the present embodiment; the receiving function 60c is equivalent to a receiver according to the present embodiment; the recognizing function 60d is equivalent to a recognizer according to the present embodiment; the registering function 60e is equivalent to a register according to the present embodiment; the transmitting function 60f is equivalent to a transmitter according to the present embodiment; the notifying function 60g is equivalent to a notifier according to the present embodiment; the priority output function 60h is equivalent to a priority output according to the present embodiment; and the searching function 60i is equivalent to a searcher according to the present embodiment.

In the present embodiment, the processing circuitry 60 may be configured by a processor. Here, the word processor means a Central Processing Unit (CPU), a Graphics Processing Unit (GPU), an Application Specific Integrated Circuit (ASIC), a programmable logic device (a Simple Programmable Logic Device (SPLD), a Complex Programmable Logic Device (CPLD), or a Field Programmable Gate Array (FPGA)), or the like. The processor realizes functions by reading and executing a program stored in the memory 84. Note that, it is also possible to configure by directly incorporating the program inside the processor circuit instead of storing the program inside the memory 84. In this case, the processor realizes functions by reading and executing the program incorporated into the circuit. Note that the processor is not limited to be configured as a single circuit, but may be configured by combining multiple independent circuits to realize the function. It is also possible to combine the elements shown in FIG. 2 into one processor to realize the function.

In the embodiment shown in FIG. 2, each processing function performed in the acquiring function 60a, the list output function 60b, the receiving function 60c, the recognizing function 60d, the registering function 60e, the transmitting function 60f, the notifying function 60g, the priority output function 60h, and the searching function 60i, is stored in the memory 84 in the form of a computer executable program. The processing circuitry 60 is a processor that realizes functions related to each program by reading and executing the program from the memory 84. In other words, the processing circuitry 60 that has read each program now possess each function shown inside the processing circuitry 60 in FIG. 2. Note that, in FIG. 2, there is a single processing circuitry that realizes the acquiring function 60a, the list output function 60b, the receiving function 60c, the recognizing function 60d, the registering function 60e, the transmitting function 60f, the notifying function 60g, the priority output function 60h, and the searching function 60i inside a single processing circuitry 60, however, possible embodiments are not limited to this example. The processor may be configured by combining multiple independent processors to realize the function by letting each processor to execute each program.

An input interface 80 is a circuitry that receives various user input, and in the present embodiment, for instance, may receive an action performed on the analysis result data, a search condition used to search the analysis result data, or the like. In the present embodiment, the input interface 80 may be a mouse a keyboard, a trackball, a manual switch, a foot switch, a button, a joystick, or the like.

The display 82 displays various images or information. For instance, the display 82 may display the medical image based on the medical image data MID acquired by the imaging apparatus, analysis result data transmitted from the diagnosis support apparatus, a Graphical User Interface (GUI) to receive various user input, or the like. In the present embodiment, the display 82 may be configured by a liquid crystal display or a Cathode Ray Tube (CRT) display.

The memory 84 may be realized by a Random Access Memory (RAM), a semiconductor memory element such as a flash memory, a hard disk, an optical disk, or the like. In the present embodiment, the memory 84 may store the medical image data MID, the analysis result data, or the like.

The communication circuitry 86 implements various telecommunications protocol according to the form of the network NW. The communication circuitry 86 realizes communication with a different apparatus via the network NW according to these various protocol. Specifically, in the present embodiment, the medical image diagnosis apparatus 200 is connected to the network NW via the communication circuitry 86 to communicate with the diagnosis support apparatus 400 or the medical image storage apparatus 600.

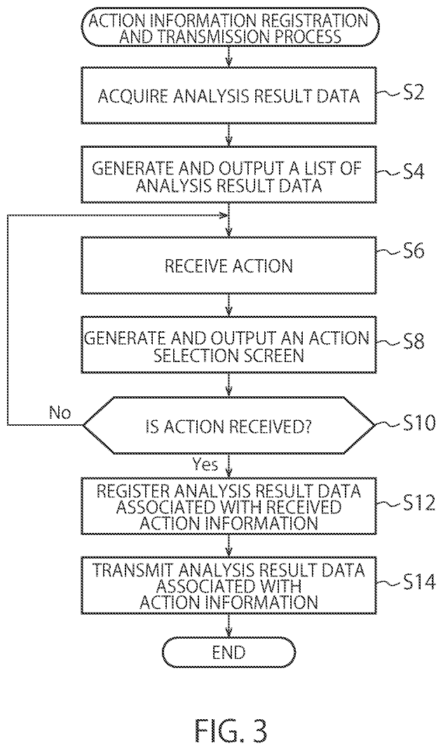

FIG. 3 is a flowchart for explaining a content of an action information registration and transmission process performed on the medical image processing apparatus 40 according to the first embodiment. In the action information registration and transmission process, the action information is registered in association with the analysis result data, and the action information associated with the analysis result data is transmitted to the different apparatus. This action information registration and transmission process may be a process that is performed when the user designates the medical image data MID and inputs a command to display a list of the analysis result data to the medical image processing apparatus 40.

As shown in FIG. 3, the medical image processing apparatus 40 first performs a process of acquiring the analysis result data (Step S2). This process of acquiring the analysis result data is realized by the acquiring function 60a in the processing circuitry 60. Specifically, the medical image processing apparatus 40 acquires the analysis result data from the diagnosis support apparatus 400 via the communication circuitry 86. The analysis result data may be directly acquired by the acquiring function 60a in the processing circuitry 60 via the communication circuitry 86 or may be acquired by the acquiring function 60a in the processing circuitry 60 from the memory 84, after storing the analysis result data acquired via the communication circuitry 86 in the memory 84.

Also, when there exists a different analysis result data related to the medical image data MID which is the source of the acquired analysis result data, the medical image processing apparatus 40 may be configured to acquire the different analysis result data as well. Note that, the different analysis result data may be an analysis result for a same patient using the diagnosis support apparatus 400B which is different from the diagnosis support apparatus 400A that was used to generate the acquired analysis result data, or an analysis result for a different disease but using the same diagnosis support apparatus 400A that was used to generate the acquired analysis result data, or an analysis result for a different disease using the diagnosis support apparatus 400B which is different from the diagnosis support apparatus 400A that was used to generate the acquired analysis result data.

Here, the process for the case when there exists the different analysis result data related to the medical image data MID which is the source of the acquired analysis result data, and when the medical image processing apparatus 40 acquires the different analysis result data, will be explained.

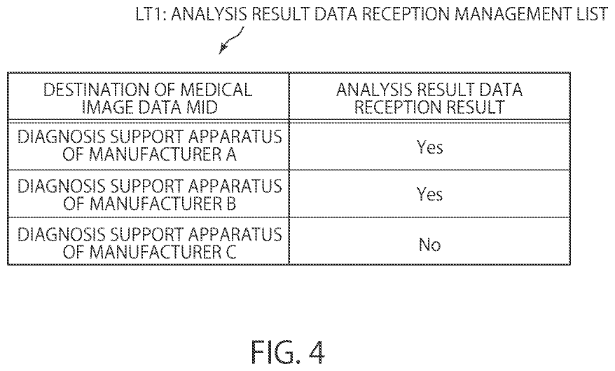

FIG. 4 is a diagram illustrating an example of an analysis result data reception management list LT1 that manages a reception result of the analysis result data associated with a destination of the medical image data MID, in the medical image processing system 1 according to the present embodiment. As shown in FIG. 4, the analysis result data reception management list LT1 stores the destination indicating which diagnosis support apparatus 400 the medical image data MID was transmitted to, associated with the reception result indicating whether the analysis result data generated based on the transmitted medical image data MID was received. In the present embodiment, the analysis result data reception management list LT1 may be stored in the memory 84.

In the example of the analysis result data reception management LT1 illustrated in FIG. 4, the medical image processing apparatus 40 indicates that the medical image data MID has been transmitted to the diagnosis support apparatus (manufacturer A) 400A, the diagnosis support apparatus (manufacturer B) 400B, and the diagnosis support apparatus (manufacturer C) 400C. Also, in the example of the analysis result data reception management LT1 illustrated in FIG. 4, the medical image processing apparatus 40 indicates that the analysis result data based on the medical image data MID generated from the diagnosis support apparatus (Manufacturer A) 400A and the diagnosis support apparatus (Manufacturer B) 400B has been received (receipt result: Yes), and that the analysis result data based on the medical image data MID generated from the diagnosis support apparatus (Manufacturer C) 400C has not been received (receipt result: No).

In FIG. 4, since there was no analysis result data received from the diagnosis support device (Manufacturer C) 400C, the acquiring function 60a in the processing circuitry 60 of the medical image processing apparatus 40 acquires the analysis result data based on medical image data MID designated by a user, from the diagnosis support apparatus (Manufacturer C) 400C via the network NW. In this case, when the analysis result data based on the medical image data MID has not yet been generated in the diagnosis support apparatus (Manufacturer C) 400C, the medical image processing apparatus 40 cannot obtain the analysis result data of the diagnosis support apparatus 400C at this point.

Next, as illustrated in FIG. 3, the medical image processing apparatus 40 performs a process that generates and outputs the list of the analysis result data (Step S4). The process that generates and outputs the list of the analysis result data is realized by the list output function 60b in the processing circuitry 60. Specifically, the medical image processing apparatus 40 extracts information about the diagnosis support apparatus 400 that was used for analysis and information about the analysis result from the analysis result data. Then, the medical image processing apparatus 40 generates the list of analysis result data from an extracted information and displays on the display 82. Here, the information about the analysis result may be the information that indicate a name of the disease that was detected, diagnosis results as positive or negative, an analysis result that quantifies the degree of positive or negative, or the like. Also, when the analysis result data has not been received, the information about the analysis result contains information that the analysis result data has not been received.

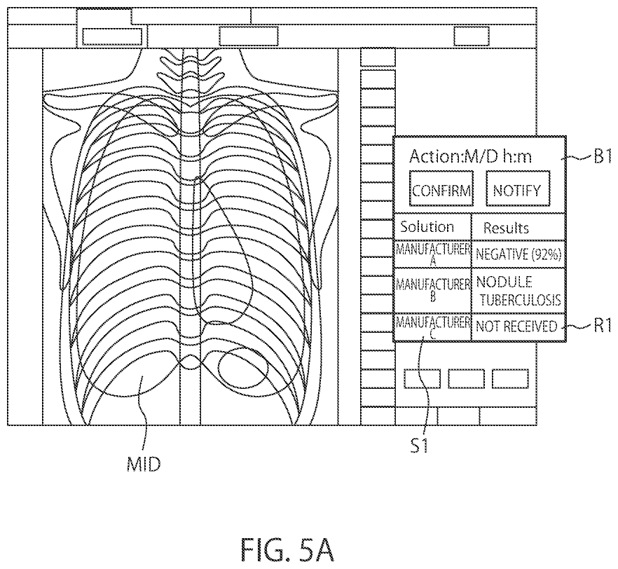

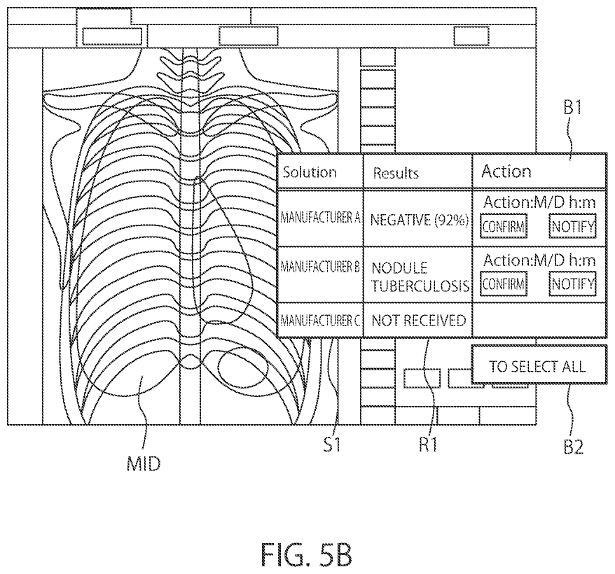

FIG. 5A is a diagram illustrating an example of the list of the analysis result data shown on the display 82 according to the present embodiment. As shown in FIG. 5A, the display 82 displays an information S1 about which diagnosis support apparatus 400 has performed analysis and an information R1 about the analysis result. In the example of FIG. 5A, the list of the analysis result data displays that the analysis result of the diagnosis support apparatus (Manufacturer A) 400A is negative (92%), that the analysis result of the diagnosis support apparatus (Manufacturer B) 400B is a nodule and tuberculosis, and that the analysis result of the diagnosis support apparatus (Manufacturer C) 400C has not been received. Note that the list of the analysis result data may be displayed with the medical image based on the medical image data MID. In the example of FIG. 5A, an X-ray image of the subject's chest is displayed as the medical image based on the medical image data MID.

Also, the medical image processing apparatus 40 may be configured to switch between show or hide the list of analysis result data based on the user operation via the input interface 80. For example, the user may switch the icon shown on the display 82 between show or hide by operating via the input interface 80. Furthermore, the medical image processing apparatus 40 may be configured to output the list of analysis result data to the memory 84 instead of the display 82, or to both the display 82 and the memory 84.

Then, as illustrated in FIG. 3, the medical image processing apparatus 40 performs a process that receives an action about the analysis result data (Step S6). This process of receiving the action is realized by the receiving function 60c in the processing circuitry 60. Specifically, when the user performs a necessary action on the analysis result data, the medical image process apparatus 40 receives a selected user action as the action information. Here, the action may be confirming the analysis result data, notifying a different user, the user isolating the patient, or the like. The action information may be the information about the user action. Note that, the information about the user action may include time information such as date and time of when the action was performed, or moreover, other additional information. The additional information may be a log or a free content such as a memo about an operation result, a confirmation result or a notification record about the action selected and performed by the user, or information such as image or text data about the selected user action.

Also, the medical image processing apparatus 40 may automatically recognize the action based on user action performed on the medical image processing apparatus 40. The process of automatically recognizing the action is realized by the recognizing function 60d in the processing circuitry 60.

Also, the action is not limited to user actions. In other words, the action may be performed by the medical image diagnosis apparatus 200 or the medical image processing apparatus 40 based on the analysis result data. The medical image processing apparatus 40 may be configured to receive the action automatically performed by the medical image diagnosis apparatus 200 or the medical image processing apparatus 40 as the action information. For example, in Step S2, when the medical image processing apparatus 40 acquires the analysis result data that there is a possibility of tuberculosis, the medical image processing apparatus 40 may automatically notify a related information terminal such as a nurse center based on the analysis result data. Then, the medical image processing apparatus 40 may receive the notifying action automatically selected by the medical image processing device 40 as the action information.

Furthermore, when the medical image processing apparatus 40 automatically selects and performs the action, the medical image processing apparatus 40 automatically recognizes action based on the communication result with the different apparatus connected to the network NW. The process of automatically recognizing the action is realized by the recognizing function 60d in the processing circuitry 60. Specifically, for instance, when the medical image processing apparatus 40 notifies the related information terminal such as the nurse center based on the analysis result data, the medical image processing apparatus 40 recognizes the notify based on the communication result between the information terminal and the medical image processing apparatus 40.

Next, as shown in FIG. 3, the medical image processing apparatus 40 performs a process that generates and outputs an action selection screen (Step S8). The process that generates and outputs the action selection screen is realized by the receiving function 60c in the processing circuitry 60. Specifically, as shown in FIG. 5A, the medical image processing apparatus 40 may generate a button B1 for typical actions such as confirm or notify as the action selection screen and display on the display 82. In the example of FIG. 5A, the medical image processing apparatus 40 generates and outputs a selection screen of the one action for the multiple analysis result data. Therefore, by selecting the one action the user is able to select the action for the multiple analysis result data at once. Note that the medical image processing apparatus 40 may display a text input screen on the display 82 instead of generating the button B1 as the action selection screen.

FIG. 5B is a diagram illustrating a different example of the action selection screen displayed on the display 82 according to the present embodiment. As shown in FIG. 5B, the display 82 may display a list indicating the information S1 about the diagnosis support apparatus 400 which performed the analysis, the information R1 about the analysis result, and the button B1 as the action selection screen for each analysis result. In other words, the user may select different actions for each analysis result. In the example of FIG. 5B, the medical image processing apparatus 40 does not display the action selection button B1 for the diagnosis support apparatus (Manufacturer C) 400C for which the analysis result data has not been received, but displays an action selection button B1 for the diagnosis support apparatus (Manufacturer A) 400A and the diagnosis support apparatus (Manufacturer B) 400B for which the analysis result data has been received, allowing an action selection.

Also, as shown in FIG. 5B, the display 82 may display a switch button B2 that switches between the action selection screen which allows to select the action for each analysis result and the action selection screen which allows to select the action for the multiple analysis result data at once. The user may operate the switch button to switch between the action selection screen which allows to select the action for each analysis result and the action selection screen which allows to select the action for the multiple analysis result data at once. Note that the switch button B2 may be in a form other than the button.

Next, as shown in FIG. 3, the medical image processing apparatus 40 determines a receipt of action for the analysis result data (Step S10). When the receiving function 60c in the processing circuitry 60 has not received the action (Step S10: No), the medical image processing apparatus 40 returns to Step S6 and repeats the process of receiving the action for the analysis result data (Step S6) and the process of generating and outputting the action selection screen (Step S8).

On the other hand, when the receiving function 60c in the processing circuitry 60 has received the action (Step S10: Yes), the medical image processing apparatus 40 performs a process that associates and registers the analysis result data and the received action information (Step S12). The process of associating the analysis result data and the action information and registering is realized by the registering function 60e in the processing circuitry 60. Specifically, the medical image processing apparatus 40 associates the analysis result data and the action information received by the receiving function 60c in the processing circuitry 60, and registers by storing in the memory 84.

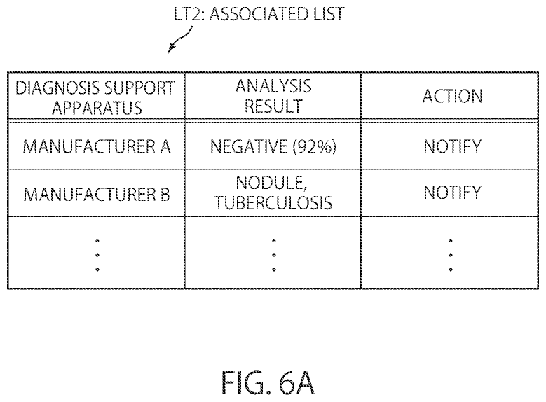

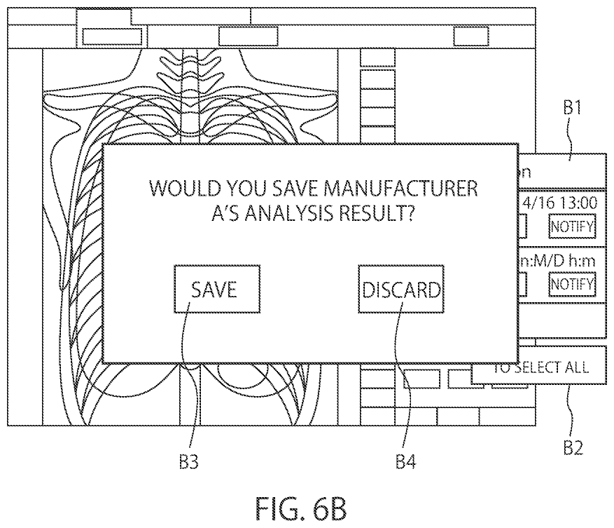

FIG. 6A is a diagram illustrating an example of an associated list LT2 of the analysis result data with the action information according to the present embodiment. As shown in FIG. 6A, the medical image processing apparatus 40 may associate “Negative (92%)”, which is the analysis result of the diagnosis support apparatus (Manufacturer A) 400A, with the received action information “Notify” and register. Then, the medical image processing apparatus 40 may associate “nodule” and “tuberculosis”, which is the analysis result data of the diagnosis support device (Manufacturer B) 400B, with the received action information “Notify and register. Then, the medical image processing apparatus 40 may associate the associated list LT2 illustrated in FIG. 6A with the medical image data MID, and register by storing in the memory 84. In other words, the associated list LT2 and the medical image data MID that images the patient are associated, registered, and stored.

Note that, in Step S10, when the receiving function 60c receives the selected action “Confirm,” which confirms the analysis result data based on the user operation, as the action information, the medical image processing apparatus 40 may display a confirmation screen confirming whether to save or discard the analysis result data for which the action was selected and enable saving or discarding the analysis result data for which the action was selected. FIG. 6B is a diagram illustrating an example of a confirmation screen shown on the display 82 according to the present embodiment. For example, as shown in FIG. 6B, the display 82 may show a message “Would you save manufacturer A's analysis result?” on the confirmation screen, along with a save button B3 and a discard button B4. Note that the message on the confirmation screen of FIG. 6B displays a message related to the analysis result of manufacturer A, but the contents of the message is not limited to this example. In other words, the contents of the message may be anything.

In the confirmation screen, when the user chooses to save the confirmed analysis result data via the input interface 80, the medical image processing apparatus 40 associates the analysis result data and the action information about the “Confirm” action received by the receiving function 60c in the processing circuitry 60 and registers by storing in the memory 84. On the other hand, when the user chooses to discard the confirmed analysis result data via the input interface 80, the medical processing apparatus 40 deletes the confirmed analysis result data and stores the additional information, which is the log that the analysis result data has been deleted, as the action information, in the memory 84.

Note that, when the user chooses to discard the confirmed analysis result data via the input interface 80, the medical image processing apparatus 40, without deleting the confirmed analysis result data, may be configured to associate the analysis result data which is the “Confirm” action received by the receiving function 60c in the processing circuitry 60 with the action information which is the log that the analysis result data has been deleted, and register by storing in the memory 84.

Next, as illustrated in FIG. 3, the medical image processing apparatus 40 performs a transmission process that transmits the analysis result data and the action information associated with the analysis result data to the different apparatus (Step S4). The process that transmits to the different apparatus is realized by the transmitting function 60f in the processing circuitry 60. Specifically, the medical image processing apparatus 40 transmits the associated list LT2 illustrated in FIG. 6A to the different apparatus. Here, the different apparatus is an apparatus connected to the network NW, such as the medical image storage apparatus 600 or a terminal device that displays analysis result data.

Note that, the process of transmitting the associated list LT2 to the different apparatus in Step S14 may be omitted if unnecessary. In this case, the associated list LT2 is stored in the medical image processing apparatus 40. A need to transmit the associated list LT2 to the different apparatus may be arbitrarily commanded by the user by an input to the medical image processing apparatus 40 via the input interface 80.

Moreover, the medical image processing apparatus 40 may be configured to refer the analysis result data reception management list LT1 shown in FIG. 4 at any time and transmit the analysis result data and the action information associated with the analysis result data to the different apparatus at Step S14, when all the analysis result data of the medical image data MID has been received. The example shown in FIG. 4 may be configured to transmit the associated list LT2 to the different apparatus when the analysis result data is received from the diagnosis support apparatus (Manufacturer C) 400C and is prepared.

Also, in Step S14, the information about the associated list LT2 shown in FIG. 6A is not limited to be transmitted to the different apparatus in the form of data. For instance, the associated list LT2 may be transmitted as image information to the different apparatus.

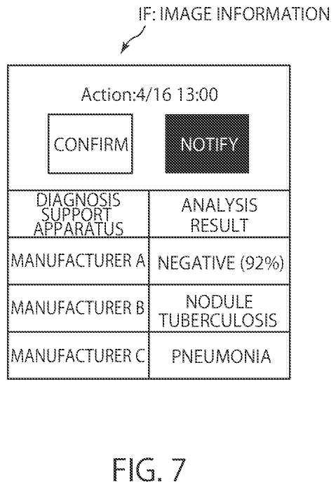

FIG. 7 is a diagram illustrating an example of an image information IF, when transmitting the list of analysis result data and the action information associated with the list of analysis result data to the different apparatus as the image information IF, according to the present embodiment. As shown in FIG. 7, the medical image processing apparatus 40 generates a Secondary Capture (SC) image for the list of analysis result data and the action information and transmits as the image information IF to the different apparatus. The list of the analysis result data shown in the image information IF of FIG. 7 indicates that the analysis result of the diagnosis support apparatus (Manufacturer A) 400A is negative (92%), the analysis result of the diagnosis support apparatus (Manufacturer B) 400B is nodule and tuberculosis, and the analysis result of the diagnosis support apparatus (Manufacturer C) 400C is pneumonia. Also, the example of FIG. 7 indicates that the performed user action based on the analysis result data is “Notify”, and that the time for which the user has performed the action is “ 4/16 13:00.”

Here, the SC image may be an image data different from the original medical image data imaged by a modality such as the medical image diagnosis apparatus 200, for example, which may be a newly generated image based on the result of external processing on the original medical image data, a capture of a displayed image, or the like.

By performing Step S14, the action information registration and transmission process according to the present embodiment is terminated.

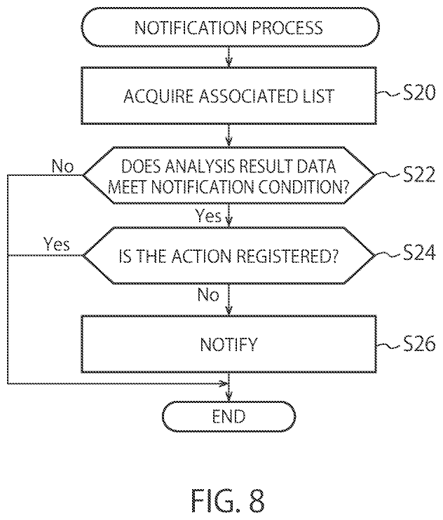

Next, the notification process that extracts the analysis result data for which the action has not been registered and notifies to the user, performed by the medical image processing apparatus 40 according to the present embodiment, will be explained. FIG. 8 is a flowchart for explaining the content of notification process performed on the medical image processing apparatus 40 according to the present embodiment. In the present embodiment, the notification process may be a process that is automatically performed when the user identifies the medical image data MID and displays on the display 82.

As shown in FIG. 8, the medical image processing apparatus 40 first performs a process that acquires the associated list LT2 from the memory 84 (Step S20). The process that acquires the associated list LT2 is realized by the notifying function 60g in the processing circuitry 60. Note that, when acquiring the associated list LT2, the medical image processing apparatus 40 may perform Step S2 in the action information registration and transmission process shown in FIG. 3 again and may additionally acquire the analysis result data from the diagnosis support apparatus 400.

Then, as shown in FIG. 8, the medical image processing apparatus 40 determines whether the analysis result data in the acquired associated list LT2 meet the notification condition or not (Step S22). Determining whether the notification condition is met or not is realized by the notifying function 60g in the processing circuitry 60. Here, the notification condition may be a condition of the analysis result data that must call attention by notifying the user when the action about the analysis result data has not been registered. In other words, the analysis result data that meet the notification condition means that it includes the analysis result which likely requires some sort of action by a doctor.

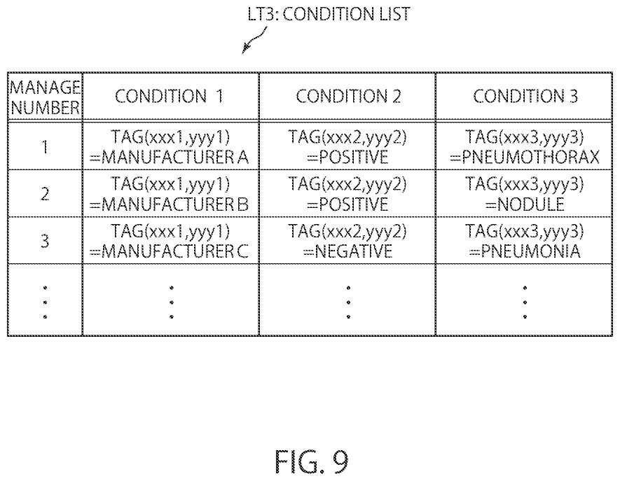

FIG. 9 is a diagram illustrating an example of a condition list LT3 for which the notification condition is registered, according to the present embodiment. In other words, when the action about the analysis result data that meet the condition, registered in the condition list LT3, has not been registered, the user is notified by the notification process. The condition list LT3 may be stored in the memory 84.

As shown in FIG. 9, according to the present embodiment, the condition list LT3 is defined using a Digital Imaging Communication in Medicine (DICOM) standard tag. The condition of the condition list LT3 is configured by a tag defined with DICOM standard (for example, TAG(xxx1, yyy1)) and a content of the data (for example, Manufacturer A) assigned to a corresponding tag. Specifically, in the example of manage number 1, “TAG(xxx1,yyy1)=Manufacturer A” is set, “TAG(xxx2,yyy2)=Positive” is set in condition 2 of manage number 1, and “TAG(xxx3,yyy3)=Pneumothorax ” is set in condition 3 of manage number 1.

Then, when the action has not been registered for the analysis result data which meet all three condition “TAG(xxx1,yyy1)=Manufacturer A,” “TAG(xxx2,yyy2)=Positive,” and “TAG(xxx3,yyy3)=Pneumothorax,” the user is notified. In other words, when all three condition is not met, there is no notification based on manage number 1.

As such, it is possible to arbitrarily set the notification condition for the user by using the condition list LT3 to determine the necessity about the analysis result data for which the action has not been registered. For example, in manage number 3, even when condition 2 of the analysis result data is negative, if condition 1 is Manufacturer C and condition 3 is pneumonia, the user receives the notification when the action has not been registered.

Note that there are three condition 1-3 set for each manage number of the condition list LT3 in FIG. 9, but the number of possible condition settings is arbitrary. In other words, the number of the condition set for one manage number may be less than two or more than four. Also, the number of condition set for each manage number may differ from each other.

Also, each condition of the condition list LT3 may be configured to be defined and stored using a data type other than the tag defined by the DICOM standard. In other words, the DICOM standard is only an exemplary format to define the condition list LT3; it is possible to configure and define the condition list LT3 using a different format.

Note that, a notification condition setting in the medical image processing apparatus 40 is arbitrary, and that it is possible to change the condition based on the user request or the user's arbitrary settings. For example, if the user sets an arbitrary condition, the condition set by the user may also be stored in the memory 84 as the condition list LT3.

Also, the notification condition does not necessarily have to be set. In this case, the process of Step S22 in FIG. 8 may be omitted. When there exists the analysis result data for which the action has not been registered, the user receives the notification unconditionally.

When the analysis result data in the acquired associated list LT2 does not meet the notification condition (Step S22: No) in Step S22 in the notification process of FIG. 8, the medical image processing apparatus 40 terminates the notification process without notifying the user. Note that, when there are multiple analysis result data, the medical image processing apparatus 40 terminates without notifying the user when all analysis result data does not meet the notification condition (Step S20: No).

On the other hand, in Step S22 in the notification process of FIG. 8, when the acquired analysis result data meet the notification condition (Step S22: Yes), the medical image processing apparatus 40 determines whether the action information about the analysis result data has been registered or not (Step S24). As described above, when there exists multiple analysis result data, if at least one analysis result data meet the notification condition, the medical image processing apparatus 40 determines whether the action information for the analysis result data has been registered or not. The process that determines whether the action information is registered or not is realized by the notifying function 60g in the processing circuitry 60.

Specifically, the medical image processing apparatus 40, based on the acquired associated list LT2, determines whether the action information for the analysis result data which meet the notification condition has been registered. Then, when the action information for the analysis result data which meet the notification condition has been registered (Step S24: Yes), the medical image processing apparatus 40 terminates without notifying the user. Note that, when determining whether the action information for multiple analysis result data which meet the notification condition has been registered, the medical image processing apparatus 40 terminates the notification process without notifying the user when the action information for the all analysis result data which meet the notification condition has been registered (Step S24: Yes).

On the other hand, in Step S24, when the action information for the analysis result data which meet the notification condition has not been registered (Step S22: No), the medical image processing apparatus 40 notifies the user that the action information has not been registered for the analysis result data which meet the notification condition, as shown in FIG. 5A (Step S26). The example shown in FIG. 5A receives an action selection by displaying a pop-up screen which includes the typical action button B1 to confirm, notify, or the like, to the display 82. The process that notifies the user is realized by the notifying function 60g in the processing circuitry 60.

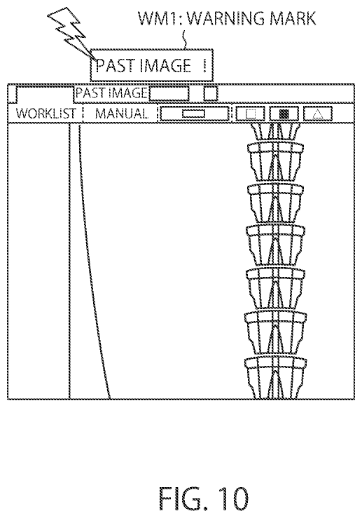

FIG. 10 is a diagram that illustrates a different embodiment of notification, displayed on the display 82 of the medical image processing apparatus 40 according to the present embodiment. As shown in FIG. 10, the medical image processing apparatus 40 may notify the user by displaying to the display 82 a notification screen that includes a warning mark WM1 and making a noise, when the action information for the analysis result data which meet the notification condition has not been registered. In other words, the notification to the user that the action information about the analysis result data which meet the notification condition has not been registered is done by both a screen display and a sound.

Note that the medical image processing apparatus 40 may notify the user by not both the screen display such as a warning mark on the display 82 and the sound, but by one of the screen display on the display 82 or the sound. Also, the medical information processing apparatus 40 may notify the user that the action information about the analysis result data has not been registered by displaying a different icon or generating a separate pop-up instead of displaying a warning mark WM1 on the display 82. Further, the medical image processing apparatus 40 may be configured to automatically start up the different application to notify the user that the action information about the analysis result data has not been registered. In other words, the embodiment of notifying the user is not limited to these examples but may be modified in various ways.

Also, as shown in FIG. 10, the medical image processing apparatus 40 may be configured to additionally display the analysis result data for which the action information has not been registered, by allowing the user to select the warning mark WM1 via the input interface 80, when the user is notified via the display 82 with a warning mark WM1 displayed on the display 82. Further, the user may individually set the contents or embodiment of the notification for each manage number of the condition list LT3 shown in FIG. 9.

By performing Step S26, by determining that the analysis result data does not meet the notification condition at Step S22, or by determining that the action information about the analysis result data which meet the notification condition is registered at Step S24, the notification process according to the present embodiment is terminated.

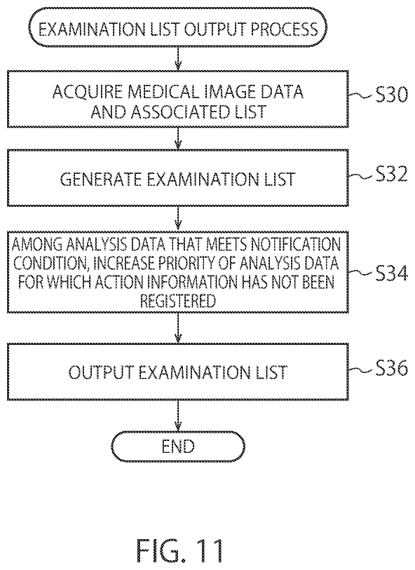

Next, a examination list output process that displays the examination list to the display 82, performed by the medical image processing apparatus 40 according to the present embodiment will be explained. FIG. 11 is a flowchart for explaining the contents of the examination list output process performed by the medical image processing apparatus 40 according to the present embodiment. The examination list output process is a process that, among the analysis result data which meet the notification condition, increases the priority of analysis result data for which the action information has not been registered to come above in the examination list when outputting the examination list. The generated examination list may be displayed on the display 82 or stored in the memory 84. Also, in the present embodiment, the examination list output process may be a process that is performed when the user commands the examination list output via the input interface 80.

As shown in FIG. 11, the medical image processing apparatus 40 first performs a process of acquiring the medical image data MID and the associated list LT2 from the memory 84 (Step S30). The process of acquiring the medical image data MID and the associated list LT2 is realized by the priority output function 60h in the processing circuitry 60. In Step S30, the medical image data MID of multiple different patients and the associated list LT2 is acquired. When the number of the acquired medical image data MID and the associated list LT2 is too large, for instance, only the medical image data MID and the associated list LT2 updated in the last week may be acquired.

Next, the medical image processing apparatus 40 generates the examination list based on the acquired medical image data MID and the associated list LT2 (Step S32). The process of generating the examination list is realized by the priority output function 60h in the processing circuitry 60. In the examination list generated in Step S30, the analysis result data of each patient and the action information about the analysis result data are simply made into the list, for example, in a random order or chronologically based on the update date and time.

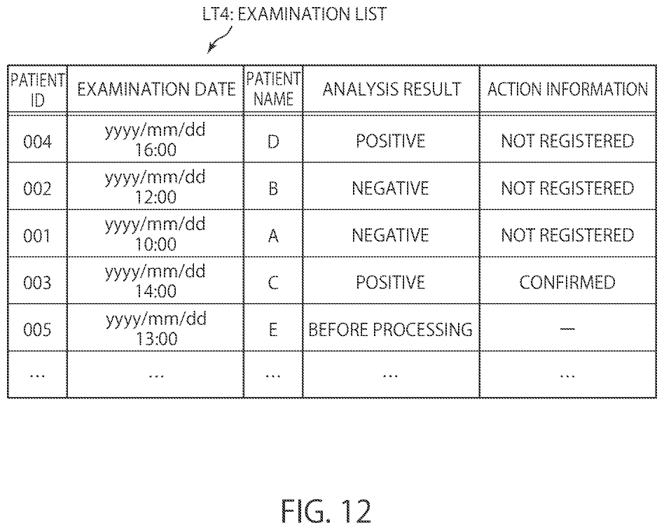

Here, FIG. 12 is a diagram illustrating an example of a examination list LT4 generated from the priority output process according to the present embodiment. As shown in FIG. 12, a patient ID, a examination date/time, the analysis result, a patient name, and the action information is associated and stored in the examination list LT4. In the present embodiment, the information about the analysis result and the action is extracted from the associated list LT2, and the information about the patient ID, the examination date and time, and the patient name is extracted from the medical image data MID which is the basis of the analysis result data. Of course, the examination list may contain other information.

Next, as shown in FIG. 11, the medical image processing apparatus 40 increases the priority of the analysis result data for which the action information has not been registered, among the analysis result data which meet the notification condition, in the generated examination list LT4 (Step S34). In other words, by increasing the priority, the analysis result data for which the action information has not been registered, among the analysis result data which meet the notification condition, is output with a high priority. In the present embodiment, to be output with a high priority means to be located above in the examination list LT4. The process of increasing the priority is realized by the priority output function 60h in the processing circuitry 60.

In the example of FIG. 12, the analysis result data which meet the notification condition but for which the action information has not been registered is located above in the examination list LT4, and all other analysis result data is located below. Also, among the analysis result data which meet the notification condition but which the action information has not been registered, the “Positive” analysis result is located relatively above in the examination list LT4.

On the other hand, for example, the analysis result data with the analysis result “Positive” and the action information “Confirmed” comes relatively below in the examination list LT4. Also, the examination which has not received the analysis result data from the diagnosis support apparatus 40 is located relatively below in the examination list LT4. In other words, in Step S34, the examination list LT4 is sorted by priority.

Note that, in the present embodiment, the reason for increasing the priority of the analysis result data for which the action information has not been registered, among the analysis result data which meet the notification condition, is because the analysis result data which meet the notification condition include analysis result which highly requires action and is presumed to require some sort of immediate action. However, in the present embodiment, it is not always necessary to meet the notification condition. In other words, the priority of the analysis result data for which the action information has not been registered may be increased and output with a high priority without determining whether the notification condition has been met.

Also, when there are multiple analysis result data for which the action information has not been registered, the analysis result data for which the action information has not been registered may be output with a high priority based on the examination date and time. Specifically, when there are multiple analysis result data for which the action information has not been registered, among the analysis result data for which the action information has not been registered, the priority of the analysis result data for which the examination date and time is old may be increased, and the analysis result data for which the action information has not been registered may be output with a high priority.

Also, when there are multiple analysis result data for which the action information has not been registered, the analysis result data for which the action has not been registered may be output with a high priority based on a type of disease or infection included in the analysis result. Specifically, when there are multiple analysis result data for which the action has not been registered, among the analysis result data for which the action has not been registered, the priority of the analysis result data which includes disease or infection which requires immediate action may be increased, and the analysis result data for which the action has not been registered may be output with a high priority. Furthermore, when there are multiple analysis result data for which the action has not been registered, the analysis result data for which the action has not been registered may be output with a high priority based on the examination date and time and the type of disease or infection included in the analysis result.

Next, as shown in FIG. 11, the medical image processing apparatus 40 outputs the examination list LT4 sorted by priority (Step S36). For instance, the medical image processing apparatus 40 may display the examination list LT4 on the display 82, transmit the examination list LT4 to the memory 84, or transmit the examination list LT4 to the different apparatus via the communication circuitry 86. The process of outputting the examination list LT4 is realized by the priority output function 60h in the processing circuitry 60.

By outputting the examination list LT4 at Step S36, the examination list output process according to the present embodiment is terminated.

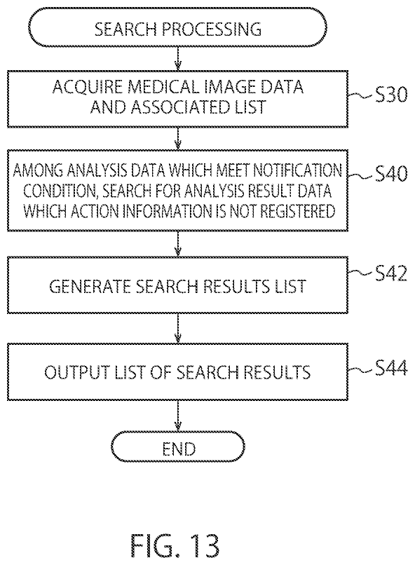

Next, a search process performed by the medical image processing apparatus 40 according to the present embodiment, that searches for analysis result data for which the action information has not been registered, will be explained. FIG. 13 is a flowchart for explaining the contents of the search process performed on the medical image processing apparatus 40 according to the present embodiment. The search process is a process that, among the analysis result data which meet the notification condition, searches for analysis result data for which the action has not been registered and outputs search results. The search process may be a process that is performed when the user commands the search of analysis result data for which the action information has not been registered via the input interface 80.

As shown in FIG. 13, the medical image processing apparatus 40 first performs a process of acquiring the medical image data MID and the associated list LT2 from the memory 84 (Step S30). Step 30 is a similar process to Step S30 in the examination list output process explained above but is realized by the searching function 60i in the processing circuitry 60.

Then, as shown in FIG. 13, the medical image processing apparatus 40 searches for, among the analysis result data which meet the notification condition, the analysis result data for which the action has not been registered based on the associated list LT2 (Step S40). The process of searching for the analysis result data for which the action has not been registered is realized by the searching function 60i in the processing circuitry 60. Specifically, the medical image processing apparatus 40 extracts analysis result data which meet the notification condition defined in the condition list LT3 shown in FIG. 9 from the acquired associated list LT2, and furthermore, extracts the analysis result data for which the action has not been registered among the extracted analysis result data.

Next, the medical image processing apparatus 40 generates the search result list based on the acquired medical image data MID and the associated list LT2 (Step S42). The process of generating the search result list is realized by the searching function 60i in the processing circuitry 60.

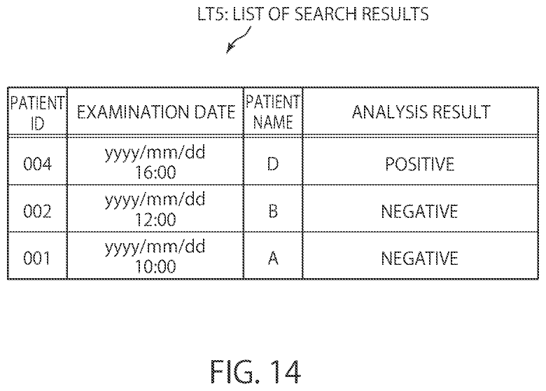

FIG. 14 is a diagram illustrating an example of the search result list LT5 generated from the search process according to the present embodiment. As shown in FIG. 14, among the analysis result data which meet the notification condition, the analysis result data for which the action has not been registered is extracted in the search result list LT5. In the example of FIG. 14, the patient ID, the examination date and time, the patient name, and the analysis result are associated and stored in the search result list LT5. In the present embodiment, the analysis result is extracted from the associated list LT2, and the information about the patient ID, the examination date and time, and the patient name is extracted from the medical image data MID which is the basis for the analysis result data. Of course, the search result list LT5 may contain other information.

Next, as shown in FIG. 13, the medical image processing apparatus 40 outputs the generated search result list LT5 (Step S42). For example, the medical image processing apparatus 40 may display the search result list LT5 on the display 82, store the search result list LT5 on the memory 84, or transmits the search result list LT5 via the communication circuitry 86 to the different apparatus. The process of outputting the search result list LT5 is realized by the searching function 60i in the processing circuitry 60.

By outputting the search result list LT5 at Step S44, the search process according to the present embodiment is terminated.

As described above, according to the medical image processing system 1 of the present embodiment, since the medical image processing apparatus 40 receives user action for the analysis result data and associates and registers the analysis result data and the action information about the action, in addition to acquiring the analysis result data from the diagnosis support apparatus 400, the medical image processing apparatus 40 of the medical image diagnosis apparatus 200 can collectively manage the analysis result data and the action information. As a result, the convenience of the user using the medical image processing apparatus 40 and the diagnosis accuracy can be increased. Also, since the medical image processing apparatus 40 can transmit the analysis result data and the action information associated with the analysis result data and registered to the different apparatus via the network NW, the user of a different apparatus which is destination can easily grasp what action has been performed on the analysis result.

Furthermore, since the medical image processing apparatus 40 can display multiple analysis result data for an arbitrary medical image data MID as the list to the display 82, the user of the medical image processing apparatus 40 can grasp the analysis result at a glance. Also, since the medical image processing apparatus 40 efficiently prompts the user to perform action when there exists the analysis result data for which the action has not been performed, an action leak for the analysis result data which require action can be evaded.

### Second Embodiment

In the medical image processing system 1 according to the first embodiment described above, the imaging apparatus 20 and the medical image processing apparatus 40 is combined to configure the medical image diagnosis apparatus 200, however, the imaging apparatus 20 and the medical image processing apparatus 40 does not necessarily have to be combined. Therefore, in the second embodiment, the medical image processing system 1, where the medical image processing apparatus 40 and the medical image diagnosis apparatus 200 are separated, will be explained. Below will be the explanation on the differences from the first embodiment described above.

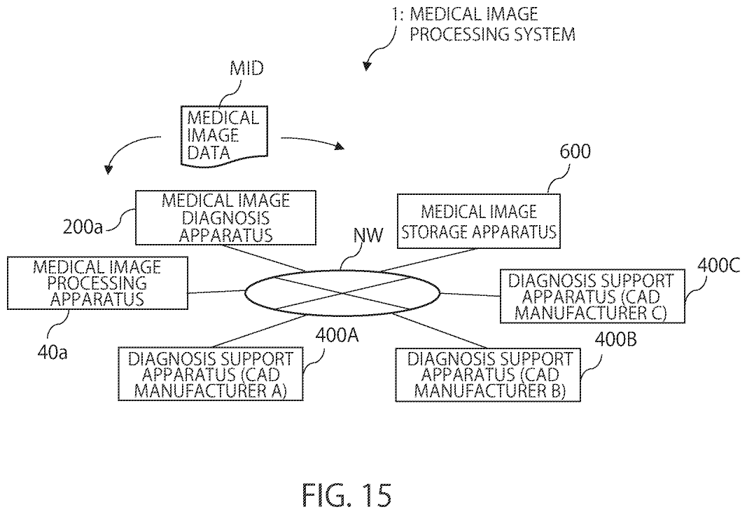

FIG. 15 is a block diagram illustrating an exemplary configuration of the medical image processing system 1 according to the second embodiment, which corresponds to FIG. 1 of the first embodiment described above. The medical image processing apparatus 40a is provided separately from the medical image diagnosis apparatus 200a as shown in FIG. 15, and the medical image processing apparatus 40a is connected to the different apparatus via the network NW.

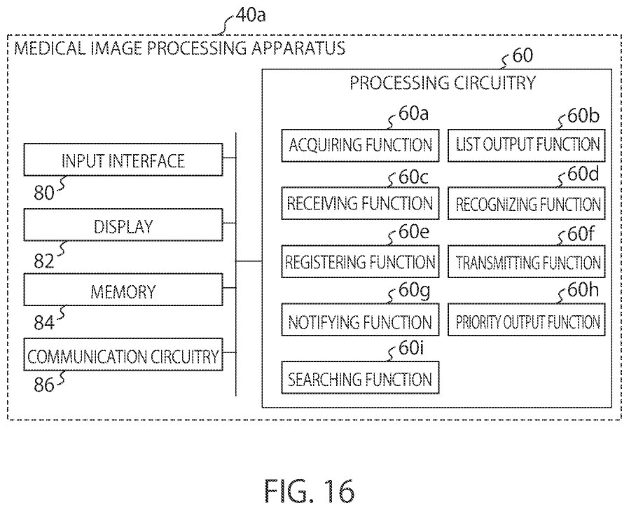

FIG. 16 is a block diagram illustrating an exemplary configuration of the medical image processing apparatus 40a according to the second embodiment. As shown in FIG. 16, the medical image processing apparatus 40 in the medical image diagnosis apparatus 200 of the first embodiment shown in FIG. 2 is configured as an independent apparatus according to the present embodiment. As such, the configuration of the medical image processing apparatus 40a shown in FIG. 16 is similar to the configuration of the medical image processing apparatus 40 in the first embodiment described above.

However, since the medical image processing apparatus 40a according to the second embodiment does not equip the imaging apparatus 20, the medical image processing apparatus 40a acquires the medical image data MID from the medical image diagnosis apparatus 200a via the network NW. For instance, the priority output function 60h in the processing circuitry 60 acquires the medical image data MID from the medical image diagnosis apparatus 200a in Step S30 in the examination list output process shown in FIG. 11. Similarly, the searching function 60i in the processing circuitry 60 acquires the medical image data MID from the medical image diagnosis apparatus 200a in Step S30 in the search process shown in FIG. 13.

In other words, in the medical image processing system 1 according to the second embodiment, since the medical image processing apparatus 40a is separate from the medical image diagnosis apparatus 200a which equips the imaging apparatus 20, the medical image processing apparatus 40a must acquire the medical image data MID from the different apparatus which equips the imaging apparatus 20. For this reason, the medical image processing apparatus 40a may acquire the medical image data MID from the medical image storage apparatus 600 instead of from the medical image diagnosis apparatus 200a.

On the other hand, the medical image diagnosis apparatus 200a in the medical image processing system 1 according to the second embodiment may be configured identically or differently from the medical image diagnosis apparatus 200 shown in FIG. 2 of the first embodiment described above. Even when the medical image diagnosis apparatus 200a shown in FIG. 15 is configured identically to the medical image diagnosis apparatus 200 of FIG. 2, the analysis result data generated based on the medical image data MID acquired from the medical image diagnosis apparatus 200 and the action for the analysis result data are collectively managed in the second embodiment at the medical image processing apparatus 40a.

On the other hand, when the medical image diagnosis apparatus 200a shown in FIG. 15 is configured differently from the medical image diagnosis apparatus 200 of FIG. 2, the medical image diagnosis apparatus 200a shown in FIG. 15 does not necessarily have to include the function of the medical image processing apparatus 40 as described in the first embodiment. In other words, the function of the medical image processing apparatus 40 may be omitted.

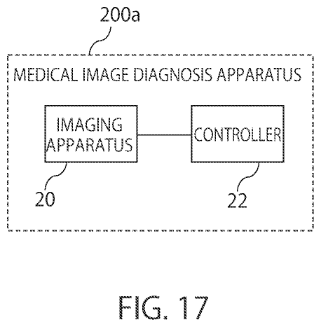

FIG. 17 is a diagram illustrating an exemplary configuration of the medical image diagnosis apparatus 200a when the medical image diagnosis apparatus 200a does not include the medical image processing apparatus 40, as described above in the first embodiment. As shown in FIG. 17, the medical image diagnosis apparatus 200a is configured to include the imaging apparatus 200 described above and a controller 22 which controls the imaging apparatus 20. Then, under a control of the controller 22, the medical image data MID acquired by imaging the subject with the imaging apparatus 20 is transmitted to the diagnosis support apparatus 400, generating the analysis result data. The generated analysis result data is transmitted to the medical image processing apparatus 40a, and the various processing described above in the first embodiment is realized in the medical image processing apparatus 40a. In other words, the analysis result data and the action information performed about the analysis result data are associated, registered, and managed in the medical image processing apparatus 40a.

As such, even in the medical image processing system 1 according to the second embodiment, similar to that of the first embodiment, since the medical image processing apparatus 40a acquires the analysis result data of the diagnosis support apparatus 400, receives the action for the analysis result data, and associates and registers the analysis result data and the action for collective management, the convenience of the user and the diagnosis accuracy can be increased.

Modified Example of the First and the SECOND EMBODIMENT

The diagnosis support apparatus 400 of the first and the second embodiment described above, may analyze the medical image data MID and suggest a recommended action based on the analysis result. In this case, the analysis result data generated by the diagnosis support apparatus 400 includes the recommended action. For this reason, for example, the acquiring function 60a in the processing circuitry 60, at Step S2 in the action information registration and transmission process of FIG. 3, may also acquire the information about the recommended action by acquiring the analysis result data.

In this case, the medical image processing apparatus 40, 40a are also able to determine whether the action is the recommended action when the receiving function 60c in the processing circuitry 60 receives the user action during the action information registration and transmission process. Then, the medical image processing apparatus 40, 40a may warn the user if the user action is different from the recommended action.

Also, in the notification process of FIG. 8 described above, the notifying function 60g in the processing circuitry 60 may notify the user that the action information has not been registered by suggesting the information about the recommended action at Step S26. By configuring in this way, the user is able to easily select an optimal action for the analysis result.

Furthermore, the medical image processing apparatus 40, 40a may automatically select the recommended action included in the information about the recommended action when the information about the recommended action is acquired, and the receiving function 60c in the processing circuitry 60 may receive the action automatically selected by the medical image processing apparatus 40, 40a as the action information.

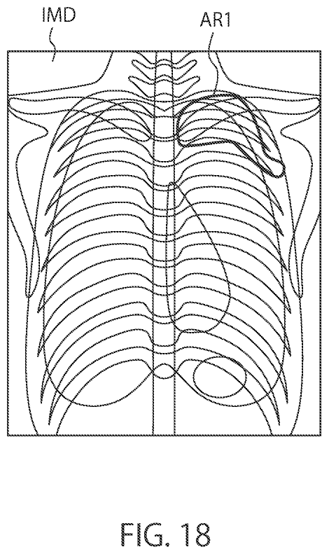

Also, the analysis result data generated by the diagnosis support apparatus 400 may include image information as well as text information. FIG. 18 is a diagram illustrating an example of image information IMD included in the analysis result data. In the image information IMD illustrated in FIG. 18, an attention mark AR1 is displayed on a portion of suspected lesion analyzed from the medical image data MID. In other words, the portion of suspected lesion is located by the diagnosis support apparatus 400 analyzing the medical image data MID. In this case, the diagnosis support apparatus 400 tags the attention mark AR1 on the portion of suspected lesion.

When the attention mark AR1 is displayed, the doctor must carefully analyze the portion which the attention mark AR is tagged. In other words, it is possible to supplementarily notify an area of the medical image which requires the doctor's special attention to realize the doctor's quick and accurate diagnosis. Also, the user, such as the doctor, becomes able to perform the optimal action for the analysis result data.

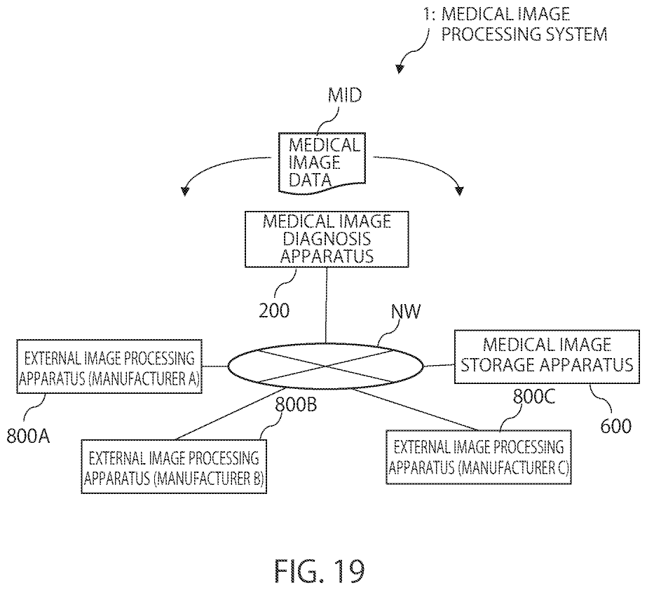

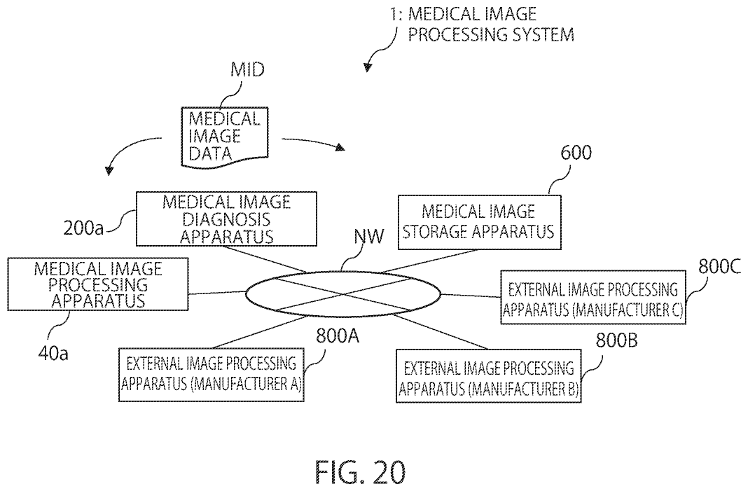

Also, the medical image processing system 1 according to the first and the second embodiment, may be configured to include an external image processing apparatus which performs the image processing as the external processing for the medical image data MID and generates an image processing data as an external processing result, instead of the diagnosis support apparatus 400 which generates the analysis result data as the external processing result. FIG. 19 is a block diagram illustrating an exemplary configuration of the medical image processing system 1 according to the modified example of the first embodiment and corresponds to FIG. 1 of the first embodiment described above. FIG. 20 is a block diagram illustrating an exemplary configuration of the medical image processing system 1 according to the modified example of the second embodiment and corresponds to FIG. 15 of the second embodiment described above.

The configuration as shown in FIG. 19 and FIG. 20 differs from that of the first and the second embodiment in that an external image processing apparatus (Manufacturer A) 800A, an external image processing apparatus (Manufacturer B) 800B, and an external image processing apparatus (Manufacturer C) 800C are included instead of the diagnosis support apparatus (Manufacturer A) 400A, the diagnosis support apparatus (Manufacturer B) 400B, and the diagnosis support apparatus (Manufacturer C) 400C. In other words, the configuration other than the external image processing apparatus (Manufacturer A) 800A, the external image processing apparatus (Manufacturer B) 800B, and the external image processing apparatus (Manufacturer C) 800C is similar to that of FIG. 1 of the first embodiment and FIG. 15 of the second embodiment described above. For this reason, detailed explanation about the configuration other than the external image processing apparatus (Manufacturer A) 800A, the external image processing apparatus (Manufacturer B) 800B, and the external image processing apparatus (Manufacturer C) 800C will be omitted. Note that, when referring to without distinction, the external image processing apparatus (Manufacturer A) 800A, the external image processing apparatus (Manufacturer B) 800B, and the external image processing apparatus (Manufacturer C) 800C, will be simply referred to as an “external image processing apparatus 800”.

The external image processing apparatus 800 performs the external processing on the medical image data MID and generates the external processing result. Specifically, for example, the external image processing apparatus 800 may perform image processing such as a bone suppression, a temporal subtraction, or a similar difference on the medical image data MID and generate an image processing result data that includes image which is image processing results, or the like. Then, the external image processing apparatus 800 transmits the generated image processing result data to the medical image diagnosis apparatus 200 or the medical image storage apparatus 600. In other words, the image processing result data is one example of the external processing result. Also, the external processing result generated by the external image processing apparatus 800 corresponds to the first medical image information of the modified example of the first and the second embodiment.

Note that, in the example of FIG. 19 and FIG. 20 in the modified example of the first and the second embodiment, the external image processing apparatus (Manufacturer A) 800A, the external image processing apparatus (Manufacturer B) 800B, and the external image processing apparatus (Manufacturer C) 800C are connected to the network NW, but the number of the external image processing apparatus connected to the network NW is not limited to this example. For example, one or two external image processing apparatus 800 may be connected to the network NW, or more than four external image processing apparatus 800 may be connected to the network NW.

As explained, the medical image processing system 1 according to the first and the second embodiment, when configured to include the external image processing apparatus 800 instead of the diagnosis support apparatus 400, it is possible to receive the user action for the image processing result data, and associate the image processing result data with the action information about the action and register, as the user acquires the image processing result data generated by the external image processing apparatus 800. For this reason, the image processing result data and the action information can be collectively managed.

Note that the medical image processing system 1 according to the first and the second embodiment is configured with the external image processing apparatus 800 instead of the diagnosis support apparatus 400, however, the medical image processing system 1 according to the first and the second embodiment is not limited to this example. The medical image processing system 1 according to the first and the second embodiment may be configured with both the diagnosis support apparatus 400 and the external image processing apparatus 800.

While certain embodiments have been described, these embodiments have been presented by way of example only, and are not intended to limit the scope of the inventions. Indeed, the novel apparatus and methods described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A medical image processing apparatus, comprising:
a memory; and
processing circuitry configured to
acquire a first medical image information, wherein the first medical image information is a result of an external processing performed on a second medical image information including a medical image,
receive a selected action for the first medical image information, and
register in the memory an action information about the selected action for the first medical image information in association with the first medical image information.

2. The medical image processing apparatus of claim 1, wherein the processing circuitry outputs a selection screen configured to select the selected action for the first medical image information and receives the selected action for the first medical image information based on a user operation.

3. The medical image processing apparatus of claim 2, wherein the selection screen is a screen where the selected action for multiple pieces of the first medical image information can be selected at once.

4. The medical image processing apparatus of claim 2, wherein the selection screen is a screen where the selected action for the first medical image information can be selected for each piece of the first medical image information.

5. The medical image processing apparatus of claim 1,
wherein the selection screen is a first screen where the selected action for multiple pieces of the first medical image information can be selected at once, or a second screen where the selected action for the first medical image information can be selected for each piece of the first medical image information; and
wherein the processing circuitry switches between the first screen and the second screen based on a user operation.

6. The medical image processing apparatus of claim 1,
wherein the processing circuitry outputs a confirmation screen configured to confirm whether to save or discard the first medical image information for which the selected action has been selected, based on the received selected action for the first medical image information; and
wherein the processing circuitry registers the action information about the selected action for the first medical image information in association with the first medical image information based on a user operation on the confirmation screen.

7. The medical image processing apparatus of claim 1, wherein the processing circuitry generates and outputs a list of the first medical image information.

8. The medical image processing apparatus of claim 1, wherein the processing circuitry transmits the first medical image information and the action information in association with the first medical image information, to a different apparatus.

9. The medical image processing apparatus of claim 1, wherein the processing circuitry outputs, among multiple pieces of the first medical image information, prioritizing the first medical image information for which the action information has not been registered.

10. The medical image processing apparatus of claim 9, wherein, when there are multiple pieces of the first medical image information for which the action information has not been registered, the processing circuitry prioritizes, based on an examination date and time and/or analysis result, the first medical image information for which the action information has not been registered.

11. The medical image processing apparatus of claim 1, wherein the processing circuitry notifies a presence of the first medical image information for which the action information has not been registered.

12. The medical image processing apparatus of claim 1, wherein the processing circuitry searches, among multiple pieces of the first medical image information, the first medical image information for which the action information has not been registered.

13. The medical image processing apparatus of claim 1, wherein the processing circuitry receives the selected action for the first medical image information as a user operation via an input.

14. The medical image processing apparatus of claim 1,
wherein the processing circuitry recognizes an action performed on the first medical image information; and
wherein the processing circuitry receives the recognized action as the selected action for the first medical image information.

15. The medical image processing apparatus of claim 14, wherein the processing circuitry recognizes the action performed on the first medical image information, based on a user operation performed on the medical image processing apparatus.

16. The medical image processing apparatus of claim 14, wherein the processing circuitry recognizes the action performed on the first medical image information, based on a communication result with a different apparatus.

17. The medical image processing apparatus of claim 1, wherein the first medical image information is generated by executing external processing performed on the second medical image information including the medical image imaged by an imaging apparatus configured to acquire the medical image by imaging a subject.

18. The medical image processing apparatus of claim 1,
wherein the first medical image information includes information about a recommended action; and
wherein the processing circuitry determines whether the recommended action has been performed or not, based on the selected action and the information about the recommended action.

19. A medical image processing system, comprising:
a diagnosis support apparatus or an external image processing apparatus configured to generate a first medical image information by executing an external processing performed on a second medical image information including a medical image; and
a medical image processing apparatus comprising a memory and processing circuitry, wherein the processing circuitry is configured to
acquire one or more pieces of the first medical image information from the diagnosis support apparatus or the external image processing apparatus,
receive a selected action for the first medical image information; and
register in the memory an action information about the selected action for the first medical image information in association with the first medical image information.

20. A non-transitory computer readable storage medium, which stores a program that causes a computer to execute processing comprising:
acquiring a first medical image information, wherein the first medical image information is a result of an external processing performed on a second medical image information including a medical image,
receiving a selected action for the first medical image information, and
registering in a memory an action information about the selected action for the first medical image information in association with the first medical image information.
